(19) United States
(12) Patent Application Publication
(10) Pub. No.: US 2012/0259233 A1
(43) Pub. Date: Oct. 11, 2012

(54) AMBULATORY PHYSIOLOGICAL MONITORING WITH REMOTE ANALYSIS

(76) Inventors: Eric K. Y. Chan, San Carlos, CA (US); Harold Strandquist, North Oaks, MN (US)

(21) Appl. No.: 13/442,300

(22) Filed: Apr. 9, 2012

Related U.S. Application Data
(60) Provisional application No. 61/473,434, filed on Apr. 8, 2011.

(51) Int. Cl. A61B 5/0205

(52) U.S. Cl. 600/484

(57) ABSTRACT

Applicants have disclosed a wireless method for remotely monitoring the physiological status of ambulatory patients by using at least one “cloud” server. Physiological data, including ECG data, is collected by a device worn by a patient and then wirelessly transmitted (e.g., via a cell phone) to the server(s). Remote processing of electrocardiograms (“ECG”) is achieved, in part, by data streaming packet lengths acquired over no less than 3 seconds—3 seconds is typically equivalent to about 3 cardiac cycles (heartbeats)—to provide the quickest response time by clinicians to try to save a heart patient's life. Other types of physiological data are monitored by the device, so medical help can be obtained when needed. In this manner, any sudden onset of vicissitudes in a patient's well being may be detected and transmitted to the care-giver and patient in near real-time.

### RELATED APPLICATIONS

This application claims priority from Applicants' U.S. Provisional Patent Application, Ser. No. 61/473,434, filed Apr. 8, 2011. Applicants claim the benefit of priority from that provisional application. Applicants also hereby incorporate the disclosure from that earlier application herein by reference.

### FIELD OF INVENTION

This invention relates in general to physiological data monitoring. More particularly, it relates to current wireless methods for remotely monitoring the physiological status of ambulatory patients.

### BACKGROUND OF THE INVENTION

Determination of the health status of medical patients is an important part of state of the art medical care. From the 19^{th} century when the deployment of early instrumentation became practical in a clinical or hospital setting, physicians began to monitor patients' vital signs to assess the need for treatment. By the beginning of the 20^{th} century, the monitoring of vital signs became a standard part of medical practice. However, physician monitoring of patient vital signs over a prolonged period of time or continuously is impractical. Yet continuous remote monitoring can be advantageous providing a comprehensive diagnostic tool useful in the context of daily living activities; or even necessary in order to discover intermittent health conditions, for example, patients at high risk for sudden cardiac death (“SCD”). Continuous remote monitoring can lead to early patient diagnosis and early therapeutic intervention which can be significantly less costly as compared with late diagnosis. Additionally, continuous remote monitoring can be useful for those conditions the measurement of which may be impacted by the presence of the patient in a clinical setting.

For example, some patients become apprehensive causing their blood pressure to rise while in the presence of medical personnel—so called, “white coat syndrome”. Providing a continuous ambulatory monitor in such cases can yield a truer measurement of the patient's health in more familiar and comfortable surroundings.

Continuous monitoring can also be advantageous in a research setting where examination of data streams can reveal phenomena that commonly precede and therefore could allow early detection of certain dangerous medical conditions.

A number of different single or dual function or even multiple function continuous monitors have become available to address needs for continuous remote medical monitoring. Most have significant drawbacks, often owing to their hardware configurations, and the need for dedicated special devices and the like. Some lack sufficient portability, and some are inconvenient. Others are not well tolerated by patients either because the devices are cumbersome or because they are so conspicuous as to stigmatize the wearer. The vast majority of currently available devices and methods continuously monitor and store patient physiologic data in the patient worn recorder with non-continuous, intermittent streaming of patient data to a single dedicated remote monitoring center. Conversely, and importantly, a distinguishing characteristic of the current invention is the capability to continuously stream data in real-time on demand to any Internet connection or if appropriate to dedicated equipment. A majority of the available monitors must store their data on-board, limiting either the resolution or the duration of monitoring and requiring lengthy data transfer and analysis sessions that are inconvenient for both doctor and patient. In some cases, the data takes as long to transfer as it does to collect; doubling the amount of time required for performing necessary diagnostics, and potentially adding risk to the patient's well-being due to delay in getting proper diagnosis. For the most part, they are designed for single mode operation requiring a patient to wear several devices if monitoring of more than one parameter is desired, and the data has to be conveyed to a processing unit or processing units and processed in turn for each of the attached devices. Therefore there is a considerable delay in multi-parameter diagnosis of physiological parameters.

U.S. Patent Application, Publication No. 2002/0124295, filed by Fenwick et al. (“Fenwick”), describes a vital signs monitor for infants undergoing “Single Room Maternity Care”. Fenwick's system uses fixed RF receptors in a hospital room to communicate with a single dedicated computer that handles data reporting, alarms and display of information. The system can infer that a patient has left a room or area within the hospital if connection to the monitor is broken. The system monitors heart rate, respiration rate, temperature, and oxygen saturation levels. While it seeks to provide vital sign and position data, it can do so only when connected to a dedicated system using dedicated hardware. Processing of the various monitored parameters cannot proceed simultaneously. This causes an inherent comparative delay in analysis because data must be analyzed in serial fashion instead of being analyzed by an array of dedicated processing servers as in the current invention. Furthermore, Fenwick's system is not capable of analyzing the heartbeat waveform for late potentials or other indicia of impending deadly arrhythmias.

U.S. Patent Application, Publication No. 2004/0215088, filed by Hubelbank, describes a cardiac monitor with a remote device communicating with the monitor in various ways. The cardiac monitor stores heartbeat and pacemaker data on an internal memory device that must be removed for processing. While it seeks to conveniently monitor and record continuous heart rate, pacemaker, and event data, it does not provide any near real-time data processing as in the current invention and there is no analysis or alarm function to alert the wearer to dangerous heartbeat wave form deviations as in the current invention. The wearer must take time to find the remote device to initiate event recording possibly exceeding the cardiac monitor's retrospective recording capacity whereas in the current invention, event recording can be started by pressing a button on the patient worn device, or by automatic event detection, including recording and file transfer to a remote central monitoring station. Processing in the prior art device and method is done using a dedicated computer system presumably in serial fashion which is inherently slower than the distributed cloud model of the current invention.

U.S. Pat. No. 7,542,878 to Nanikashvili discloses a personal health monitor and method of health monitoring enabling the acquisition, and processing of physiological data within a patient worn device with subsequent long range transmission of the data. While it seeks to provide a record of “processed” data only, there is no storage capacity for “raw” data and no capability to re-analyze the raw patient data. The processing takes place in serial fashion on the patient worn device, thus the form factor must include memory for data and processing instructions and hardware that is not required in the present invention. Furthermore, since data processing is done serially, it is comparatively slower than processing by an array of dedicated process servers as in the present invention.

Accordingly, it is a general object of the present invention to provide a wireless method for remotely monitoring the physiological status of ambulatory patients and reporting a problem in the shortest time to medical help.

It is another general object to provide such a method for both high and low resolution heartbeat monitoring of ambulatory patients.

It is a more specific object, commensurate with the above, to provide an improved wireless method for remotely streaming high resolution ECG data files from ambulatory patients in quick packet lengths and then processing Signal-Averaged Electrocardiograms (“SAECG”) using “cloud” servers.

### SUMMARY OF THE INVENTION

Applicants have disclosed a wireless apparatus (a.k.a. system) and method for remotely monitoring the physiological status of ambulatory patients by using “cloud” servers. Remote processing of Signal-Averaged Electrocardiograms (“SAECG”) is achieved, in part, by data streaming packet lengths of no less than 3 seconds—which is typically equivalent to about 3 cardiac cycles (heartbeats). Other physiological data (e.g., blood pressure, respiration, oxygen saturation) are monitored by the device, so medical help can be obtained when needed.

Applicants' preferred overall system is functionally divided into three parts. These three parts of the system carry out the acquisition, reduction, reporting and presentation of patient information.

Part one is accomplished using a lightweight multi-sensor, multi-parameter device, worn by a patient, for data acquisition and transmission. The device governs acquiring and transmitting patient information (e.g., via a cell phone over the Internet) and also receiving input in the form of instructions and alerts. The preferred device is capable of acquiring data from sphygmomanometers (blood pressure), thermometers, respiration monitors, both hi and low resolution electrocardiogram (“ECG”) sensors having any customary assortment of lead arrangements (e.g., 3, 5, 7 or 12 lead), and oxygen saturation or (SpO₂) probes, among other things.

Part two represents a “cloud”-based distributed data analysis, storage, and reporting system using one or more servers, remotely located.

Part three represents rapid methods of reporting and displaying patient data and patient alert information. After processing, servers in the cloud prepare patient information in pre-configured reports and the physician, and in appropriate cases, the patient will be notified that the reports are ready.

Applicants believe it is critical to gather packets of data streams which are no shorter than 3 seconds and to transmit those data streams immediately (i.e., a split second after acquisition by a patient worn device) in short bursts for processing to provide the quickest response time by clinicians to try to save a heart patient's life.

Three seconds typically is the equivalent of 3 heartbeats; and, in the event of tachycardia, about 10 heartbeats. Anything less than 3-second strips do not provide an accurate rhythmic strip of ECG data for analysis; e.g., a comparison of the previous 3-seconds of data with the present 3-seconds of data permits rapid automated detection of changes in morphology and rate of the electrocardiograms.

### BRIEF DESCRIPTION OF DRAWINGS

The above and other objects and advantages of the present invention will become more readily apparent upon reading the following description and drawings in which:

FIG. 1 depicts an overview of the three basic functions of Applicants' preferred method and apparatus for “Ambulatory Physiological Monitoring with Remote Analysis”;

FIG. 2 depicts a functional block diagram of: a preferred patient worn device which detects and transmits patient parameters and receives programming and alert information; and one manner of transmitting digital data from individual monitoring devices through a digital multiplexer;

FIG. 3 depicts Applicants' preferred embodiment of translating analog data from multiple inputs into a 16 Bit digital data stream;

FIG. 4, labeled “PRIOR ART”, depicts how current conventional ambulatory physiological monitoring devices communicate with a dedicated server;

FIG. 5 depicts how Applicants' preferred ambulatory physiological monitoring system communicates with a private cloud server;

FIG. 6 depicts an exemplary computer dialog box wherein a user can select from a list of parameters to monitor or to request reports;

FIG. 7 depicts a typical data stream produced using Applicants' method and apparatus;

FIG. 8 depicts a second example of a typical data stream, produced using Applicants' method and apparatus, allowing the longer part of the data stream to be transmitted and processed separately;

FIG. 9 depicts how multiple inputs can be sorted and combined to form a short burst data packet;

FIG. 10 depicts a flow of parallel packet data to an administrative server and then on to an array of dedicated application servers for processing;

FIG. 11 depicts a functional block diagram of a cloud server with raid memory and flow of processed data reports to a thin client terminal on demand; and

FIG. 12 depicts an exemplary signal path for optional on demand real-time continuous streaming of ECG data.

### DETAILED DESCRIPTION OF THE PREFERRED EMBODIMENT(S)

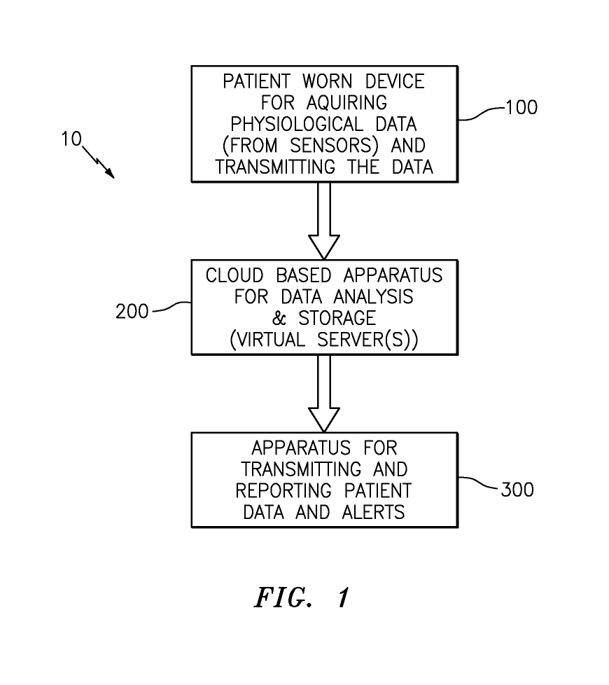

Referring to FIGS. 1-3 and 5-12, Applicants have disclosed a wireless apparatus (a.k.a. system) and method for remotely monitoring the physiological status of ambulatory patients by using one or more “cloud” servers. Remote processing of electrocardiograms (“ECG”) is achieved, in part, by streaming packet lengths of data acquired over no less than 3 seconds—which is typically equivalent to about 3 cardiac cycles (heartbeats)—and transmitting each packet individually immediately for processing (i.e., a split second after acquisition by a patient worn device) to provide the quickest response time by clinicians to try to save a heart patient's life.

As used herein, the term “clinician” refers to a physician or other qualified person who is involved in the treatment and observation of living patients, as distinguished from one engaged in research.

The cardiac cycle is the sequence of events that occurs when the heart beats. There are two phases of the cardiac cycle. In the diastole phase, the heart ventricles are relaxed and the heart fills with blood. In the systole phase, the ventricles contract and pump blood to the arteries. A typical cardiac cycle lasts about one second.

Applicants' preferred method and apparatus allow continuous, including real-time continuous streaming, simultaneous wireless acquisition of relevant patient physiologic parameters and health information; rapid, remote, near-real-time analysis, storage, and reporting of the information; alerting physicians when certain anomalous conditions are detected; and alerting patients by voice, text, and or audible or other signal to the need for patient action(s) including the need to seek immediate medical attention. It includes analysis, for example, of patient oxygen saturation levels (SpO₂), blood pressure, temperature, and both high and low resolution heartbeat monitoring. High resolution heartbeat waveform monitoring is performed to determine patient risk for Sudden Cardiac Death (“SCD”), while low resolution monitoring yields cardiac rhythm information similar to the traditional Holter monitor. When only low resolution heartbeat information is needed, the apparatus can store data for an extended period of three days to two weeks or more, in which case the near-real-time reporting functionality may not be required. The preferred method also includes provisions for patient initiated and automated event monitoring and loop recording.

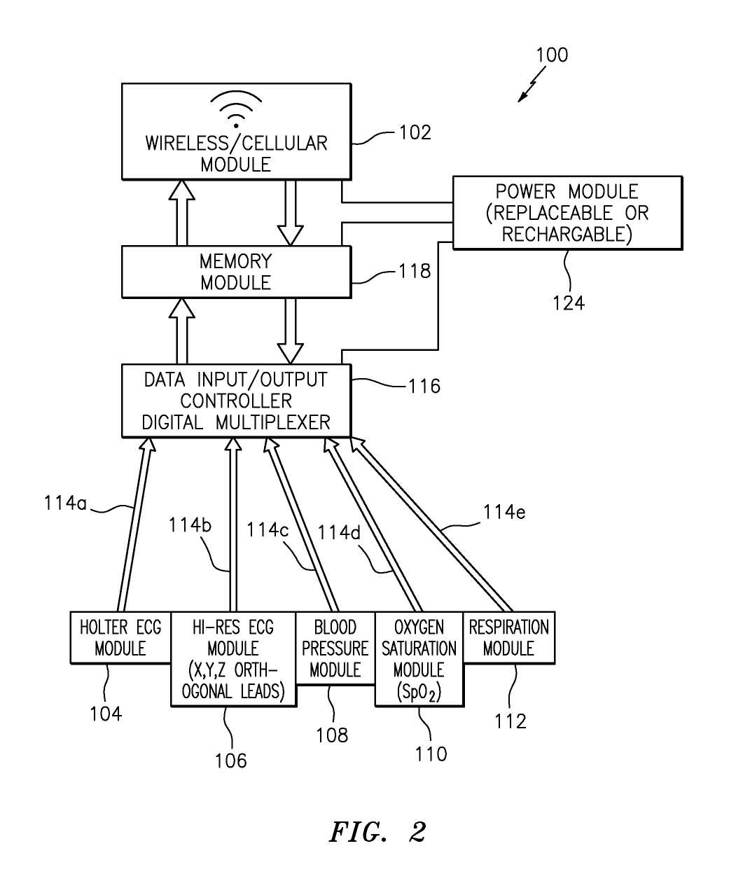

FIG. 1 is an overview of the preferred embodiment of Applicants' invention. FIGS. 2-3 and 5-12 show various individual features, as more fully described below.

Applicants' preferred ambulatory physiological monitoring system 10, with remote analysis, is functionally divided into three parts. See FIG. 1. Part one is a device 100, worn by a patient (not shown), for acquiring (via multiple sensors) physiological information from the patient and transmitting that information in short bursts. The device 100 also receives input in the form of remote parameter programming, and patient instructions and alerts. Part two (200) represents “cloud”-based distributed data analysis, storage and reporting preferably using an array of virtual servers. Part three (300) represents apparatus for reporting and transmitting patient data and patient alert information. As described more fully in the following paragraphs, the three parts (100, 200 and 300) of the system 10 fully encompass the acquisition, reduction, reporting, and presentation of patient information.

As shown in FIG. 2, the preferred patient worn device 100 takes input from any number of patient information gathering devices (e.g., the illustrated modules 104-112) including, for example, a Holter electrocardiogram (“ECG”) module 104 (e.g., 3, 5, 7, or 12 leads), a standard high resolution (“HI-RES”) ECG module 106 (e.g., 7 leads for 3 orthogonal bipolar channels and 1 reference lead), a blood pressure module 108, an oxygen saturation (“SpO₂”) module 110, a respiration module 112, and other patient information modules (not shown), for example: a temperature module, or patient position or location module.

These modules (e.g., 104, 106, 108, 110 and 112) use standard sensing devices (some downsized) to measure selected parameters and to develop an output signal. Each sensing device has an analog-to-digital converter stage after signal preconditioning and amplification stages. The hollow arrows in FIG. 2 indicate digital data bus lines 114a, 114b, 114c, 114d, 114e which feed a data input/output (“I/O”) controller digital multiplexer 116. The I/O Controller 116 stores data in memory module 118 (e.g., 8 Gigabyte).

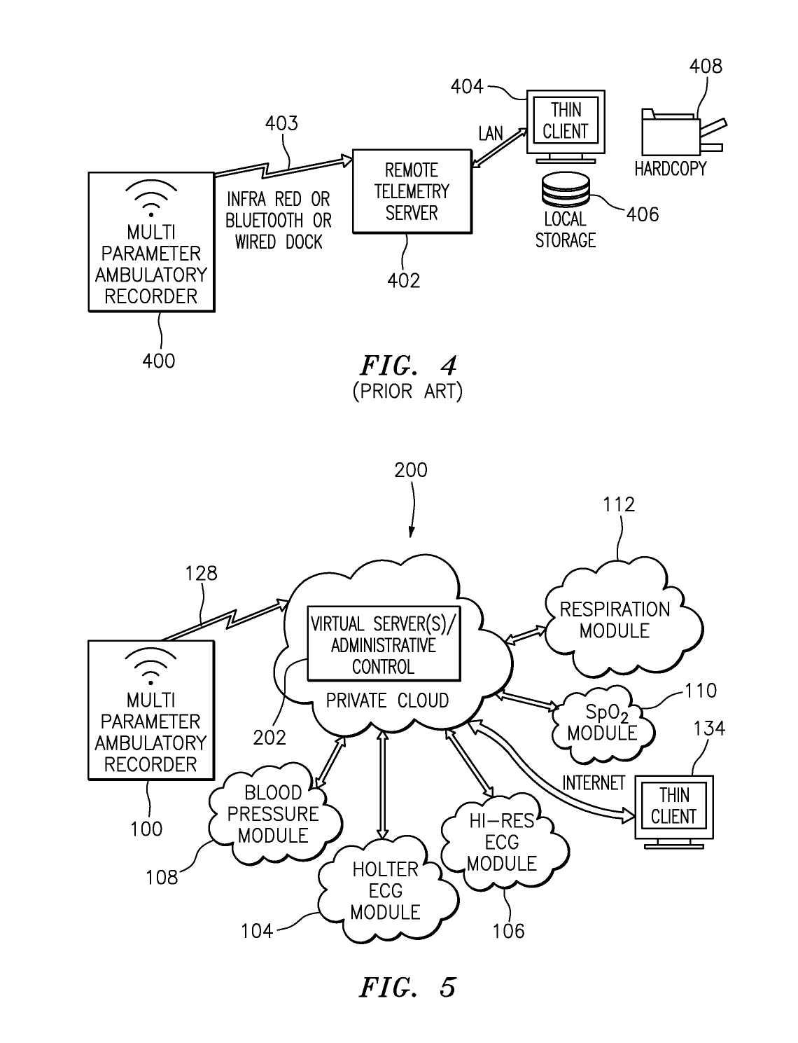

A wireless (e.g., WIFI, GPRS/3G/4G, short range wireless network) receiver/transmitter (e.g., the patient worn device 100) reads the contents of memory (in module 118), and transmits that data via a standard protocol to a remote device (e.g., telemetry server 402 in FIG. 4) which is part of a cloud based server. Input control and alert signals are received in the receiver section of the receiver/transmitter module 100.

The patient worn device 100 is so designed as to be completely portable, unobtrusive, lightweight, powered by rechargeable and replaceable batteries (see power module 124), and easy to connect and wear such that the patient's full mobility, comfort, convenience, and compliance are maximized.

The device 100 preferably comprises one or more standard electrodes (not shown), or other suitable connections, attached to the patient for the purpose of sensing the desired parameter, signal, condition, or status. The probes or connections communicate with their respective information gathering devices using either a hardwired connection or other short range electronic or optical communication devices, for example, communication means might include Bluetooth, WIFI, infra-red, Nordic or ANT. Information gathering devices can be mounted either integrally inside the patient worn device 100, or to enhance flexibility and to reduce weight, each information gathering device can be mounted detachably to the patient worn device via an auxiliary connector (not shown). Each information gathering device (module) is thus equipped with an auxiliary connector which accepts and passes through data from other information gathering devices, allowing such devices to be stacked or ganged as needed on the patient worn device.

The different types of monitoring devices, their sensors, and connecting cables if used can be color coded, or physically keyed to simplify setup, and optimize patient connection, and any combination of devices can be used at any given time.

The HI-RES ECG module 106 (see FIG. 2) is standard (but downsized). Standard HI-RES ECG devices are disclosed, for example, in various U.S. patents and patent applications including: U.S. Pat. No. 5,704,365 to Albrecht et al., issued Jan. 6, 1998, for “Using Related Signals to Reduce ECG Noise”; and U.S. Pat. No. 7,016,731 to Ryan et al., issued May 21, 2006, for “Sensing Artifact Reduction for Cardiac Diagnostic System”.

ECG is used to measure the rate and regularity of heartbeats as well as the size and position of the chambers, the presence of any damage to the heart, and the effects of drugs or devices used to regulate the heart (e.g., a pacemaker).

The HI-RES ECG module 106 is capable of capturing and recording digitally heartbeat waveforms with sufficient resolution to allow detection of very small signal transients in the microvolt level, present in the underlying waveform that are predictive of the onset of certain potentially fatal cardiac arrhythmias. As those skilled in the art will appreciate, heartbeat waveforms are characterized by peaks or waves commonly designated by letters “P,” “Q,” “R,” “S,” and “T” which correspond to electro-physical processes occurring in the heart muscle. Waveforms are aligned on P or R waves and signal averaged, in each case to inspect the waveform for different anomalies. For example, R waves are aligned and signal averaged for late potential analysis used in assessing patients at risk of sudden cardiac death, and P waves are aligned and signal averaged for prolonged P wave duration to study patients that may be at risk of atrial fibrillation. T wave variability and T wave alternans can also be analyzed with different signal processing methods that require high enough resolution to study microvolt signals.

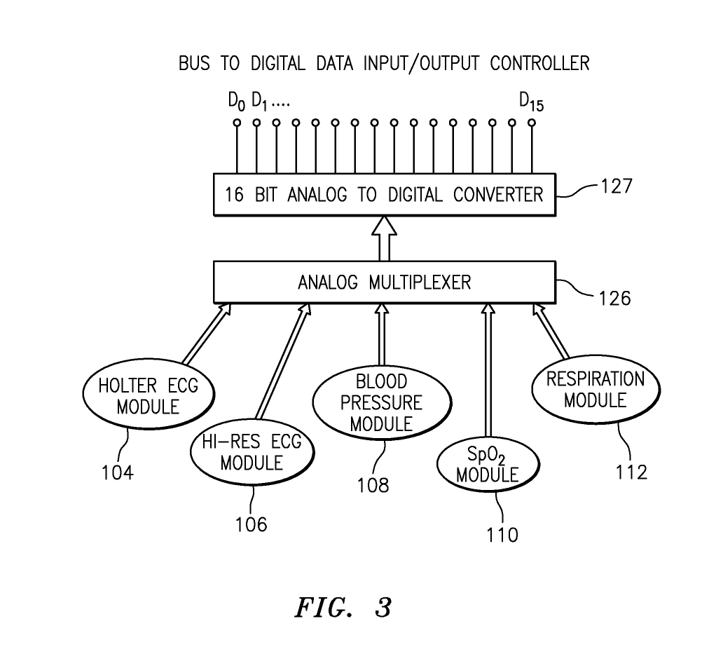

As an alternative to each patient information gathering device having its own dedicated analog-to-digital converter, a conventional analog multiplexer 126 and 16 Bit Analog-to-Digital Converter 127 (see FIG. 3) can be employed. The analog multiplexer 126 polls each information gathering device in turn using appropriate addressing logic. The multiplexer 126 then sends data including identification information to the analog-to-digital converter 127 wherein the analog signals are converted to digital signals and output on a 16 bit data bus. The 16 bit resolution of the analog to digital converter is sufficient to allow analysis of micropotentials on heartbeat waveforms and to provide sufficient detail for all other monitored parameters. While more economical, this alternative may become more complicated as additional individual patient information gathering devices with differing sampling rate requirements, latencies, etc., are added due to the analog multiplexer switching through the different incoming signals.

FIG. 4, labeled “PRIOR ART”, depicts a conventional ambulatory physiological data recorder 400. As shown, signals are sent to a dedicated remote telemetry server 402 perhaps via (at 403) infra-red, Bluetooth, or a wired docking station. Signals are sent on demand and must be processed sequentially, and then reported via a Local Area

Network (“LAN”) to a thin client (defined below) or other terminal 404, at a care-giver facility, equipped with local mass storage media 406 and provision for hardcopy output 408. The conventional method requires the patient to remain within range of the fixed remote telemetry server 402. In most cases the data must be transferred at a rate approaching the acquisition rate, which means recording and then transmitting the data takes twice as long as would transmission performed at a higher rate or at approximately the same time as the data was accumulated.

A “thin client” is an electronic communication hardware device (e.g., a terminal) which relies on a server to perform the data processing. Either a dedicated thin client terminal or a regular PC with thin client software is used to send keyboard and mouse input to the server and receive screen output in return. The thin client does not process any data; it processes only the user interface.

In Applicants' preferred embodiment, data transmission from the multi-parameter ambulatory recorder 100 is achieved via commercial-grade wireless telephony transmission modalities. Such modalities include, but are not limited to, GPRS (114 Kbps), 3G (384 Kbps) or 4G (1.3 Mbps to 7.2 Mbps) and higher data rate cellular networks as they become available.

Applicants believe it is critical to gather data streams acquired over a minimum of 3 seconds for the reasons set forth as follows. The lower end duration of 3 seconds is a minimally reasonable time to facilitate physician analysis. For example, a Normal Sinus Rhythm (“NSR”) in the approximate range of 60 beats per minute (“bpm”) would yield a 3-beat display, called a “rhythm strip”. Three heartbeats is a reasonable minimum view. In a tachyarrhythmia case, with a rate on the order of 200 bpm, a 3-second strip would show about 10 beats.

Data can be stored on the resident non-volatile memory for post-facto transmission, or transmitted immediately after collection in bursts of programmable length of no less than 3 seconds. Short bursts minimize the amount of data lost if a burst is lost due to a transmission or reception error. Shorter bursts also will maximize battery service life on the recorder thereby improving patient compliance since the unit will require less battery maintenance and less time attached to a power source. A trained physician is able to adequately diagnose an urgent care situation with this amount of data. When not performing real-time data streaming, bursts longer than 4 seconds could potentially increase the time needed to analyze the data and more importantly, issue any necessary alerts. For example, in a case in which cardiac arrest has occurred or in which analysis indicates a heightened risk for sudden cardiac death, alerts must be issued promptly to allow appropriate medical intervention. Notwithstanding immediate life threatening arrhythmias, physicians may wish to program the system (on demand) by selecting the option to remotely monitor continuous real-time streamed patient physiologic data to watch a patient's rhythm similar to in-hospital methods (see FIG. 12). In addition to the burst transmissions, the system's digital loop recorder stores at most the past 30 minutes worth of data and the contents of the loop recorder can be preserved separately on demand by the user depressing a button on the patient worn device.

With regard to the duration limits of data streaming, Applicants' device shall have burst lengths programmable in 3 or 4 seconds. Comparisons of such short bursts of consecutive data (e.g., 2 consecutive bursts of 3-seconds each) are more than sufficient to enable most physicians and even automated algorithms to diagnose problems with the heartbeat.

As shown in Applicants' FIG. 5, data is transmitted over the Internet (at 128) to one or more virtual servers in a cloud arrangement 200 (see FIG. 5). Generally in computing, the concept of a “cloud” involves computation, software, data access, and storage services that do not require end-user knowledge of the physical location and configuration of the system delivering the services. Applicants' cloud-based servers 200 use encryption and HIPAA-compliant secure storage. However, details about the number and location of servers and file storage, maintenance and so forth may not be of concern to the end users. It is intended that the method of this application could be implemented by a third party that would attend to details required for support of the cloud architecture, such as providing redundant storage, backup, and processing capabilities. The data acquisition system has to be pointed to the correct Universal Resource Listing (“URL”), and an administrative server 202 in the cloud system 200 does the rest. The administrative server(s) 202 is responsible for separating the data from each of the individual patient information devices 104, 106, 108, 110, 112 and for transmitting that data to the appropriate dedicated servers 204, 206, 208, 210, 212 (described below).

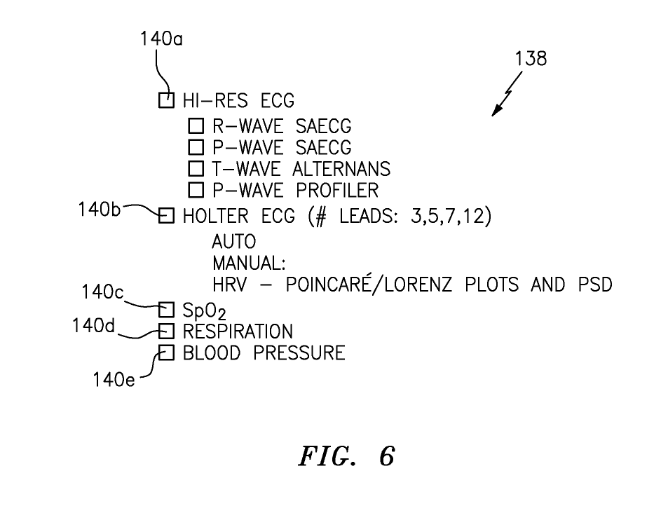

The cloud-based server system 200 provides separate processing capability for each of the patient information parameters monitored (see FIGS. 2, 6). This is done preferably by assigning dedicated servers for each type of analysis. (Alternatively, one server could be used.) Data transfer, processing, and storage is quick and efficient, simultaneous and parallel and so reports can be generated in near-real-time and optional heartbeat display functionality can be implemented as a continuous real-time stream. Processed data and reports can be viewed when ready over the Internet on any suitable Internet-ready browser including a thin client terminal 134 (see FIG. 5) or an Apple® iPhone® 135, or iPad® or any Android® Tablet PC or smart phone running Android® 2.2 (Froyo), 2.3 (Gingerbread), 3.0 (Honeycomb) 4.0 (Ice Cream Sandwich), 5.0 (Jelly Bean) or similar or later developed operating systems. It is intended that patient reports be stored in the cloud 200 and not on client systems. PDF copies of reports can be generated and printed if needed for permanent storage in the clinician's records. Record retrieval from the cloud archive can be set-up on a pay-per-view arrangement. Clinicians would access records using an interactive dialog, such as a computer display (e.g., see FIG. 6), whereupon the clinician places a check mark in the box for each desired report.

The method also allows users to select which parameters are monitored either through software, or by physically manipulating controls on the multi-parameter ambulatory recorder 100. FIG. 6 represents a sample computer display 138, an “Analysis Selection Display.” The display 138 allows clinicians to select from a list of analyses to be run on an individual patient. The clinician selects an analysis to run by placing a check in a checkbox, e.g., such as depicted boxes 140a, 140b, 140c, 140d, 140e. The same or a similar dialog box can be employed for either selection of analyses or for reporting.

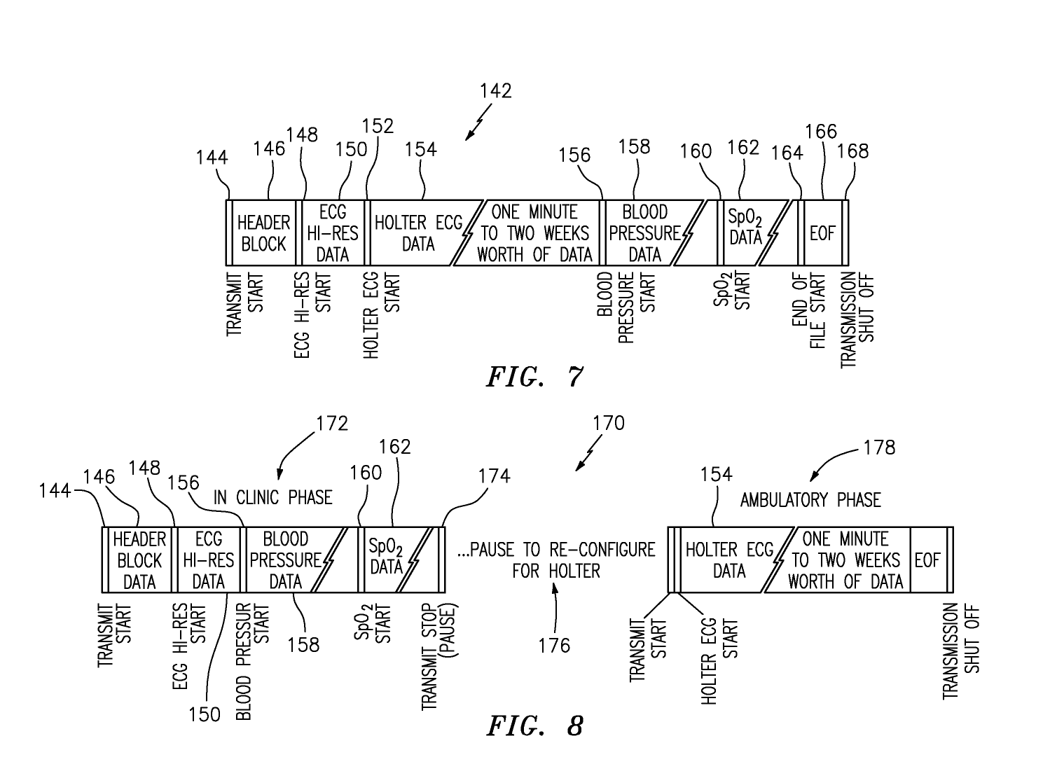

Data bursts are set to 3 seconds in length by default. As shown in FIG. 7, a typical data stream 142 (using Applicants' invention) would start with a transmit start signal (at 144) followed by a header 146 containing, for example, the patient's demographic information, name, age, sex, height, weight, and address. When transmission of the header is complete an ECG high resolution start signal 148 is transmitted followed by the ECG high resolution data stream 150. Next the Holter ECG Start signal (at 152) is transmitted followed by Holter heart monitoring data 154 that can range from one minute to two weeks in length. The Holter data takes a relatively long period of time to transmit. Next, at 156, a blood pressure start signal is sent followed by the stream 158 of blood pressure data. Next the start signal 160 for the oxygen saturation (SpO₂) measurement is sent along with SpO₂ data 162, after which other data (not shown; e.g., temperature) can be measured and sent, and finally, the end-of-file start and end-of-file signals 164, 166 are sent. At the end of the burst a transmission shut off signal is sent identifying the end of the transmission 168.

In the preferred embodiment, the data stream 168 (depicted in FIG. 7) can be improved slightly by placing the Holter monitor data 154 at the end of the improved stream 170 allowing the other information to be extracted first and processed. FIG. 8, which shows the improved data stream 170, also provides the ability to perform split monitoring—i.e., monitoring of HI-RES ECG data 150, blood pressure 158, oxygen saturation 160 in the clinical setting 172 and then switching to Holter ECG data 154 only mode for ambulatory purposes. In the later case a transmit stop signal (at 174) is sent following the oxygen saturation 160, and after the multi-parameter ambulatory recorder 100 is reset and paused (at 176), the Holter monitor data 154 is sent (e.g., during an ambulatory stage 178). When only Holter monitoring is being performed, three days to two weeks' worth of data may be stored on the server.

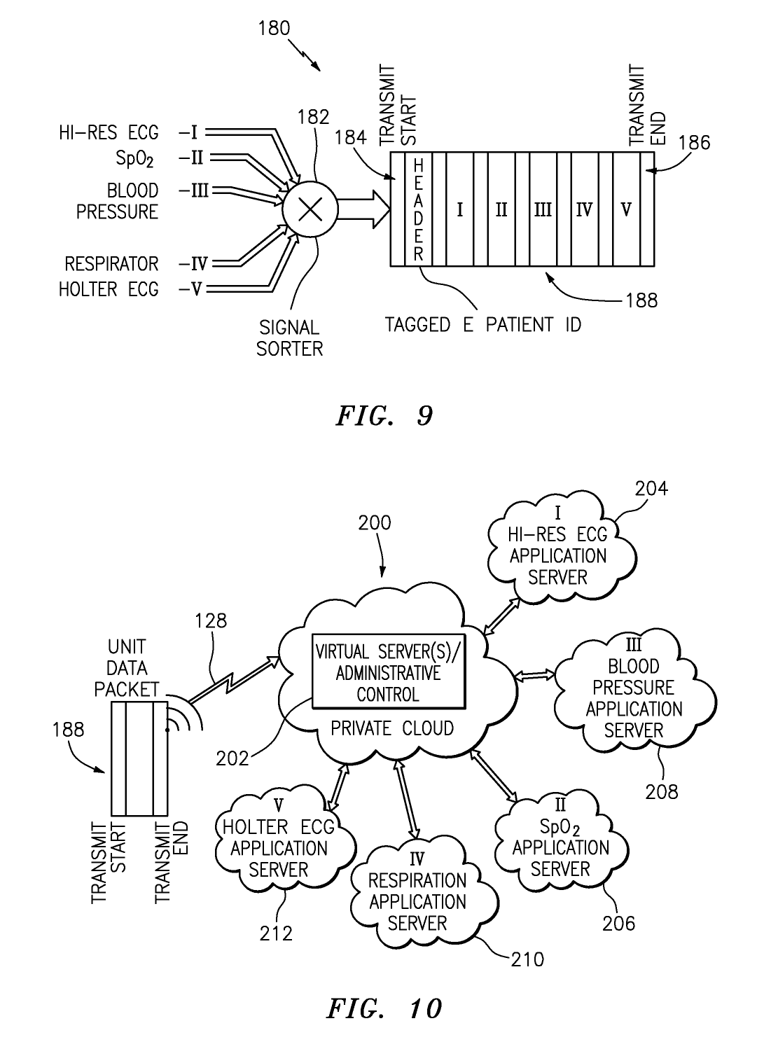

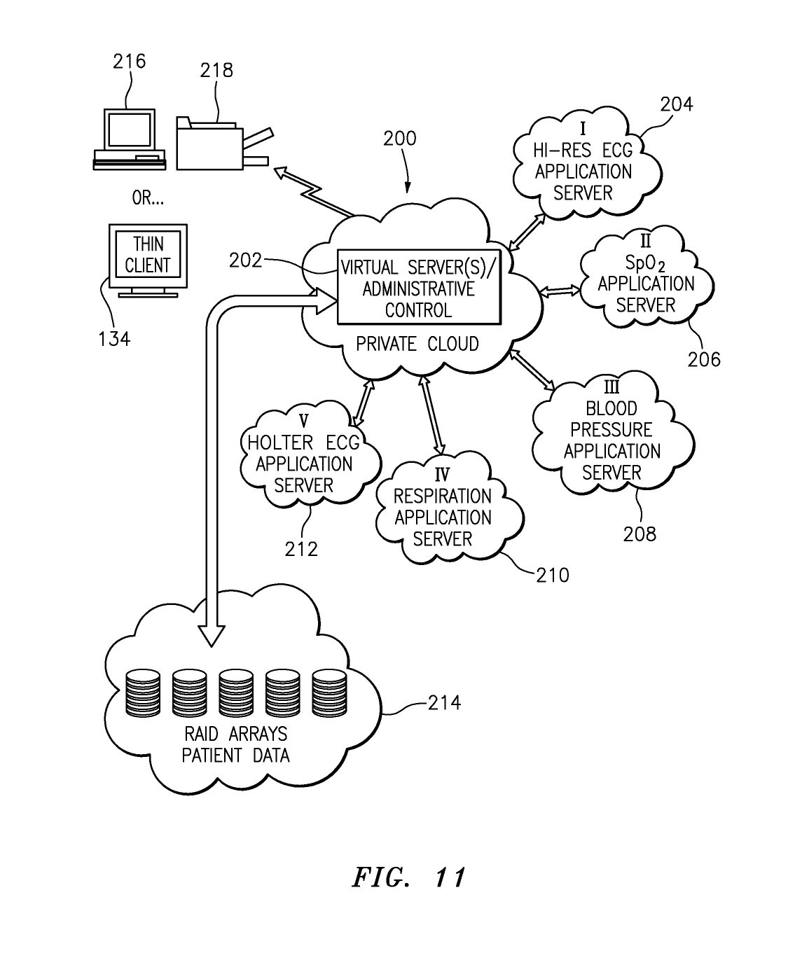

The method employed to prepare data for burst transmission 180 is shown in FIG. 9. A burst of data is shown in FIG. 10.

Referring to FIG. 9, data from each burst are processed through a signal sorter or scrambler 182. The scrambler 182 inserts the necessary start signal and stop signals 184, 186 and inserts data segments to form “parallel packets” in the proper order, and the scrambler 182 adds a check sum or other data integrity checking mechanism. The data for each burst, in this FIG. 9, is stored as a single unit—a “unit data packet” 188.

The process is reversed when data arrives at the virtual administrative server 202 in the cloud-based system 200 (FIG. 10). This is a unit data concept with distributed signal processing on dedicated application servers. In it, individual parallel packets (e.g., 188) are transmitted by one or more administrative servers 202 to individual processing servers such as the illustrated HI-RES application server 204, SpO₂ application server 206, blood pressure application server 208, respiration application server 210 and Holter ECG application 212. There, the data packets are processed according to the source of the data. The order of data, the kind of data transmitted, and types of patient physiology data contained in the data stream presented in the foregoing discussion are exemplary and not intended to limit the variability of types of information that could be obtained, analyzed, stored, and reported.

By analyzing the patient's physiological data in short bursts of, for example, 3-second data packets, and then separately checking each type of vital signs (e.g., cardiac rhythm, blood pressure, oxygen saturation, respiration) on individual processing servers, any sudden change in those vital signs can be detected. For example, the lack of any heartbeat can be detected quickly, as can tachycardia, shallow breathing, a spike in blood pressure, or a sudden drop. Life-threatening events give rise to the administrative server 210 alerting a clinician to send a first response team immediately. In addition, automated algorithms may be employed to compare information in the previous 3-second data packet with the presently incoming 3-second data packet, to detect sudden changes within a near real-time scenario and automatically sending an alert.

As those skilled in the art will appreciate, packet communication protocols such as those used in the current invention and those used over the internet and digital telephony systems typically rely on the transmission and reception of packets which are often divided into three parts usually referred to as the header, the payload, and the trailer. The header takes care of synchronization, and informs the receiving node as to the overall packet length and the position of the packet within a stream of packets. The payload carries the data. The trailer contains the cyclic redundancy check or checksum used by the receiving node to confirm that the packet was received correctly. If a packet is not received correctly, the receiving node discards the packet and submits a re-send request to the transmitting node.

The preferred packets of the current invention can either be transmitted or received directly or they can reside within the payload section of the packets of any other communication protocol, thus enabling the use of any suitable commercially available communication equipment.

Among the data contained in preferred packets, is High Resolution (“HI-RES”) Electrocardiographic data which is processed to find ventricular late potentials in the ECG by R Wave Signal Averaged ECG (“SAECG”); it is processed to study atrial fibrillation patients using P wave SAECG and other P wave parameter analyses. T Wave

Alternans analysis may also be applied on High Resolution ECG data using dedicated digital signal processing and analysis methods. The latter analysis can predict patients' risk for Sudden Cardiac Death (“SCD”) by predicting the onset of ventricular tachycardia, for example. Of course, the absence of a heartbeat can also be detected and the system can dispatch the appropriate alert messages.

As mentioned above, Applicants have found that it is critical to use short packet bursts of ECG data acquired over a minimum of 3 seconds. Any suitable known method of Signal Averaged Electrocardiography would suffice, provided the method could analyze packet lengths of 3 seconds of ECG data.

Signal-averaged electrocardiography (“SAECG”) applies computerized ensemble averaging of electrocardiogram (“ECG”) complexes during sinus rhythm to detect microvolt signals called ventricular late potentials, used in the risk stratification of patients at risk of sudden cardiac death due to re-entrant ventricular tachycardia (“VT”). SAECG data acquisition requires a stable signal environment in order for the auto-templating process to successfully identify candidate beats. “Sub-optimal” high resolution electrocardiograms (“HI-RES ECG”) are characterized by the presence of noisy signals or intermittently changing ventricular systole (“QRS”) morphologies, along with baseline drift due to respiratory artifacts and patient movement.

Arrhythmia Research Technology, Inc. (“Arrhythmia Research”), according to the assignment records of the U.S. Patent and Trademark, is the Assignee of: U.S. Pat. No. 5,025,794 to David A. Albert and Edward J. Berbari for “Method for Analysis of Electrocardiographic Signal QRS Complex” issued Jun. 25, 1991 (“Albert et al.”); U.S. Pat. No. 5,609,158 to Erik K. Y. Chan for “Apparatus and Method for Predicting Cardiac Arrhythmia by Detection of Micropotentials and Analysis of All ECG Segments and Intervals” issued Mar. 11, 1997 (“Chan”); and U.S. Pat. No. 4,422,459 to Michael B. Simson for “Electrocardiographic Means and Method for Detecting Potential Ventricular Tachycardia” issued Dec. 27, 1983 (“Simson”). The present Applicants hereby incorporate these patents in their entirety by reference.

Arrhythmia Research markets the processes in U.S. Pat. Nos. 4,422,459 (Simson), 5,025,794 (Albert et al.) and 5,609,158 (Chan) as software under the trademark PREDICTOR™.

The Chan patent, issued in 1997, disclosed apparatus (hardware) which could run the PREDICTOR™ software. As stated in Chan, at col. 8, lines 1-41, the apparatus can comprise any suitable microcomputer, such as an IBM PC, having an electrocardiographic signal acquisition unit connected thereto. Chan recites:
The computer 12 includes a system bus 16 for carrying data, address, control and timing signals between the various modules of the computer 12, all of which are well known to those skilled in the art. A microprocessor 18 is connected to the system bus 16 for communication therewith. A keyboard unit 20 and a mouse unit 21 are connected to the system bus 16 for communication therewith, as is a memory 22, comprising both read only memory (ROM) and random access memory (RAM), a video display card or graphics adapter 23 and a display 24, and a printer 26. A disk controller 28 is connected for communication with the system bus 16, and a floppy disk 30, an optical disk 31 and a hard disk 32 are connected to the disk controller 28 for communication therewith as storage devices. All of these aforementioned elements comprise the conventional microcomputer system 12.

Also connected to the system bus 16 through a detachable serial, parallel, PCMCIA data link I/O port 35 is a portable signal acquisition unit 14 which, for example may be a real time acquisition module, e.g. a model 1200EPX, or computer peripheral cards, e.g. models LP-Pac Q, Predictor I and Predictor IIc signal-averaging system, which may be obtained from Arrhythmia Research Technology, Inc., Austin, Tex. Alternatively, the signal acquisition unit 14 may include off-line mode analog or digital storage, e.g., playback of Holter recorder data. This signal acquisition unit 14 includes a microprocessor 41 having a storage device and a RAM memory 43, that is battery powered to retain contents of the storage device for extended periods. The unit 14 also contains an analog-to-digital converter 40 that is connected to a multiplexer 42, and both are controlled by the microprocessor 41. The multiplexer 42 is connected to an X-lead bipolar electrocardiographic amplifier 44, a Y-lead bipolar electrocardiographic amplifier 46, and a Z-lead bipolar electrocardiographic amplifier 48, which are adapted to be connected via respective bipolar leads X, Y and Z and a ground lead G to a patient for the sensing of electrocardiographic signals from the patient's body, as is well known to those skilled in ECG technology.

The PREDICTOR™ software, until 2010, ran on a single hardware based platform. Now it is reconfigurable for a variety of hardware platforms. The conversion allows PREDICTOR™ software to be used with customer-specific electrocardiogram acquisition equipment to generate the signal-averaged ECG.

The legacy code of PREDICTOR™ software handles seed-beat selection and template formation differently for manual and automatic modes. The PREDICTOR™ software's code (legacy DOS left unchanged in present WINDOWS® iterations) begins by reading in the first 8 beats of a RDF file. The legacy code comments say this is to “let things settle”; hence the sole purpose of this step is to skip forward 8 beats.

Then, the PREDICTOR™ software reads in the next 4 beats in sequence (beats 9, 10, 11, and 12), and uses these as seed-beat candidates. These 4 beats are cross-correlated with each other, resulting in six cross-correlations. If a pair of beats are cross-correlated to approximately 99%, then a score of 0 (zero for affirmation, i.e., negative logic) is given to each cross-correlation process.

Since Applicants' present invention calls for reviewing no less than 3 beats, PREDICTOR™ software can be modified to review every subsequent 3 (or 4) beats. For example, PREDICTOR™ could read the next 3 beats in sequence and use these as seed-beat candidates. These 3 beats can be cross-correlated with prior sets.

At least one of the processing servers in the cloud keeps a record of the ECG monitor data in a virtual loop recorder application. The loop is recorded in an administrator defined variable length (i.e., duration) record. The “retrospective” feature of a conventional event recorder is superfluous since the pre-defined variable length ECG record serves as an archive of ECG loop history. Provided there is a more or less continuous unbroken stream of data reaching the virtual loop recorder application, the ambulatory device does not even need to have resident memory on board. This architecture permits design of a very compact event recorder or wireless Mobile Cardiac Telemetry (MCT) device. However, since memory is relatively inexpensive, if form factor permits, a memory loop may operate on the patient worn device to provide more reliable or alternative loop recording functionality.

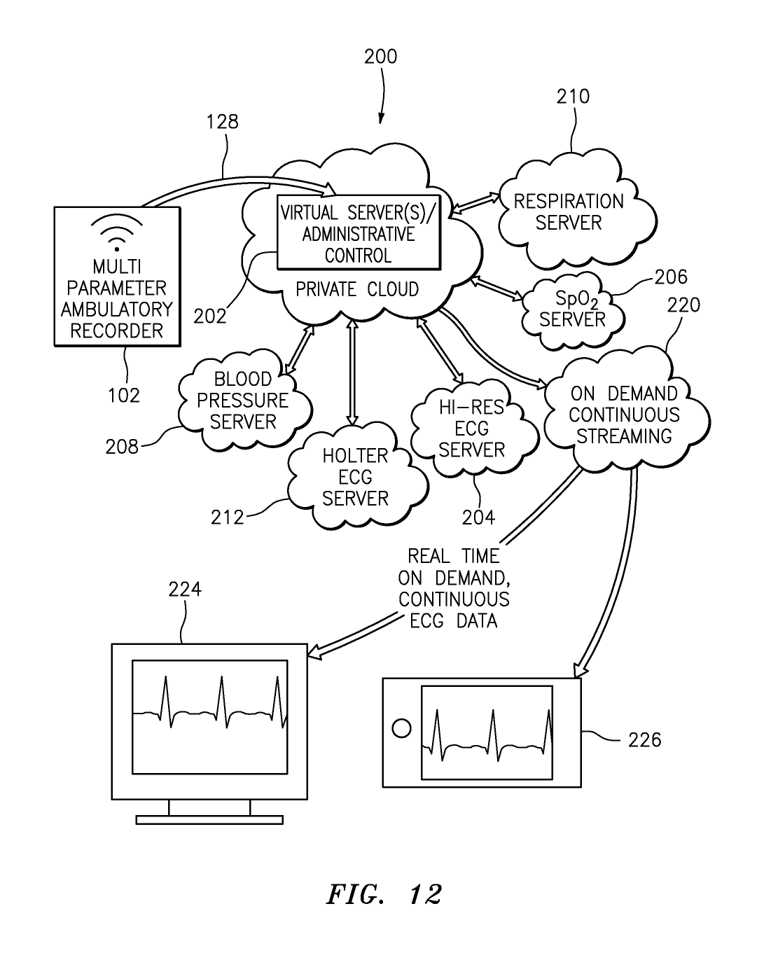

After processing in each of the respective cloud based processing servers (see FIG. 10), data is sent back to the administrative server(s) 200 to be collated, stored and formatted into report format (see FIGS. 11, 12). As shown in FIG. 11, the server(s) 202 stores its data digitally on a private cloud-based series of raid arrays 214 that are kept backed up, secure, and HIPAA-compliant.

The server 202 notifies the user that a report is ready by sending an e-mail or text message and furnishes the report on demand to a thin client terminal 134, full computer workstation (with a server) 216, or a printer 218 or any other Internet ready devices 226, such as an Apple® iPhone® or iPad®, or Tablet PC or smart phone running Android® 2.2 (Froyo) or 2.3 (Gingerbread) or 3.0 (Honeycomb) or similar or later developed operating systems.

As indicated at 300 in FIG. 1, the private cloud administrative operating system 200 is also capable of sending an alert to the patient worn device 100 to warn the patient of the need to seek immediate medical treatment if warranted. Similarly, the operating system will notify clinicians (e.g., via an iPhone® 226 and/or a thin terminal 224) to send or provide help. See FIG. 12.

The current invention also is capable of continuous real-time patient data streaming 133 to remote processors, if desired by a physician. Continuous streaming 220 enables real-time remote beat-to-beat monitoring capability (see FIG. 12). Consequently, in the presence of any arrhythmia, the physician may opt to view the patient's electrocardiogram in real-time remotely at central station or via a secure internet portal anywhere within Internet range, e.g., by a thin terminal 224 or iPhone® 226. Thus, the current invention will have true remote telemetry functionality.

In one sense, Applicants' preferred method of remotely monitoring the physiological status of ambulatory patients can be thought of as:

a. acquiring physiological data from the patient, on a device worn by the patient wherein:
i. the physiological data comprises packet lengths of data acquired over a minimum of 3 seconds; and
ii. each of the packet lengths comprises data about multiple types of vital signs including blood pressure, respiration and ECG data;

b. wirelessly transmitting bursts each of the packet lengths, as each of the packet lengths is acquired by the device, over the Internet to a remote, cloud-based processing server;

c. breaking down the packet lengths into separate types of vital signs upon receipt by the processing server;

d. analyzing the types of vital signs, separately on respective servers, to determine if there the patient has encountered a sudden medical condition that would require immediate medical assistance; and

e. alerting a clinician, via the server, upon determining a sudden medical condition of the patient, to provide immediate medical help to the patient.

Additional process steps can include, e.g., comparing a previous packet of physiological information with a present packet of incoming physiological information to detect sudden changes in near real-time mode, and issue alerts.

The sudden method condition can involve, e.g.: the patient's heart rate; the patient's heart rhythm morphology; the patient's breathing; the patient's blood pressure.

It should be understood by those skilled in the art that obvious modifications can be made to Applicants' preferred apparatus or related method without departing from the spirit or scope of the invention. Accordingly, reference should be made primarily to the following claims rather than the foregoing description to better understand the scope of the present invention.

We claim:

1. A method of remotely monitoring a patient comprising:
a. acquiring physiological data from the patient, on a device worn by the patient wherein:
i. the physiological data comprises packet lengths of data acquired over a minimum of 3 seconds; and
ii. each of the packet lengths comprises data about multiple types of vital signs including blood pressure, respiration and ECG data;
b. wirelessly transmitting bursts each of the packet lengths, as each of the packet lengths is acquired by the device, over the Internet to a remote, cloud-based processing server;
c. breaking down the packet lengths into separate types of vital signs upon receipt by the processing server;
d. analyzing the types of vital signs, separately on respective servers, to determine if there the patient has encountered a sudden medical condition that would require immediate medical assistance; and
e. alerting a clinician, via the server, upon determining a sudden medical condition of the patient, to provide immediate medical help to the patient.

2. The method of claim 1 further comprising: comparing a previous packet of physiological information with a present packet of incoming physiological information to detect sudden changes in near real-time mode, and issue alerts.

3. The method of claim 1 wherein the sudden medical condition involves the patient's heart rate.

4. The method of claim 1 wherein the sudden medical condition involves the patient's heart rhythm morphology.

5. The method of claim 1 wherein the sudden medical condition involves the patient's breathing.

6. The method of claim 1 wherein the sudden medical condition involves the patient's blood pressure.

7. A method of remotely monitoring an ambulatory patient comprising:
a. acquiring physiological data from the ambulatory patient, on a device worn by the patient, wherein the physiological data comprises packet lengths of ECG data acquired over a minimum of 3 seconds;
b. wirelessly transmitting individual bursts of each of the packet lengths, as each is acquired by the device, over the Internet to a remote, cloud-based processing server;
c. analyzing each of the packet lengths of ECG data for indications of a medical condition with the patient's heart; and
d. alerting a clinician, via the server, upon determining an indication of a medical condition with the patient's heart.

8. The method of claim 7 further comprising: comparing a previous packet of physiological information with a present packet of incoming physiological information to detect sudden changes in near real-time mode, and issue alerts.

9. The method of claim 7 further comprising:
a. analyzing each of the packet lengths of ECG data, immediately upon receipt by the server, to determine any absence of a heartbeat of the patient; and
b. expeditiously alerting medical practitioners, via the server, upon determining an absence of a heartbeat, to send medical help to the patient.

10. The method of claim 7 further comprising: upon determining an indication of arrhythmia, sending an alert to the device worn by the patient to warn the patient to seek medical treatment.

11. A method of remotely monitoring an ambulatory patient comprising:
a. acquiring physiological data from the ambulatory patient, on a device worn by the patient, wherein the physiological data comprises packet lengths of data acquired over a minimum of 3 seconds;
b. wirelessly transmitting bursts each of the packet lengths, as each of the packet lengths is acquired by the device, over the Internet to a remote, cloud-based processing server;
c. analyzing each of the packet lengths of physiological data, immediately upon receipt by the server, to determine if the patient has encountered a sudden medical condition that would require immediate medical assistance; and
d. expeditiously alerting a clinician, via the server, upon determining the sudden medical condition of the patient, to send medical help to the patient.

12. The method of claim 11 wherein the sudden medical condition is cardiac arrest.

13. The method of claim 11 wherein the sudden medical condition involves cardiac rhythm.

14. The method of claim 11 wherein the sudden medical condition involves blood pressure.

15. The method of claim 11 wherein the sudden medical condition involves breathing.
